(19) United States
(12) Patent Application Publication
(10) Pub. No.: US 2026/0183561 A1
(43) Pub. Date: Jul. 2, 2026

(54) WEARABLE PHOTOTHERAPY MASK

(71) Applicant: Shenzhen Kaiyan Medical Equipment Co., Ltd., Shenzhen (CN)

(72) Inventors: Alain Dijkstra, Amstelveen (NL); Chen Shoufeng, Shenzhen (CN); Wu Guoqiu, Shenzhen (CN)

(73) Assignee: Shenzhen Kaiyan Medical Equipment Co., Ltd., Shenzhen (CN)

(21) Appl. No.: 19/435,615

(22) Filed: Dec. 29, 2025

(30) Foreign Application Priority Data
Dec. 31, 2024 (CN) 2024233214674

(51) Int. Cl. A61N 5/06; A61L 31/06

(52) U.S. Cl. CPC A61N 5/0616; A61L 31/06; A61N 5/0617; A61N 5/0624; A61N 2005/0626; A61N 2005/0647; A61N 2005/0652; A61N 2005/0659; A61N 2005/0663

(57) ABSTRACT

The present invention relates to a phototherapy face mask system configured to conform closely to a user’s facial contours for enhanced comfort and uniform light delivery. The mask body comprises a bendable sheet with two eye-opening regions, each incorporating an interior slit and reinforced connecting positions that guide a tension strap. When tightened, the strap draws adjacent mask edges inward to minimize bulging and air gaps. The light-emitting assembly includes a translucent bottom film, a flexible light panel, and a backlit top film, the films extending beyond the panel and being ultrasonically welded to seal and protect the electronics. LEDs are positioned outside clearance zones aligned with the interior openings to prevent compression during folding. A control unit, connected through a strain-relieved cable interface, supplies power and control signals and includes a GPS module, an electrophoretic display for adaptive operation and status indication.

### TECHNICAL FIELD

The present disclosure relates to wearable phototherapy devices for skin treatment, and more particularly to a flexible facial phototherapy mask system designed to conform closely to a user's facial contours through a strap-tensioned structure with a sealed multilayer light panel assembly for enhanced comfort and uniform therapeutic light delivery.

### BACKGROUND ART

Phototherapy, also known as light therapy, involves the application of low-level light energy to stimulate or regulate biological processes with therapeutic effects. LED phototherapy utilizes light-emitting diodes to deliver beneficial light energy from the visible and infrared portions of the light spectrum to the skin. Specific wavelengths of light interact with biological systems and activate cellular receptors, triggering a transfer of light energy to cellular energy. This process can stimulate skin repair mechanisms, promote cell renewal, and address various skin conditions. LED phototherapy has gained recognition as a modality for skin care applications and is used for the treatment of inflammatory skin conditions, wound healing, and skin rejuvenation.

Phototherapy masks have been developed as wearable devices for delivering light therapy to a user's face. These masks typically comprise a body structure having an LED array disposed on an interior surface to irradiate the facial skin with therapeutic light. Some existing phototherapy masks employ rigid or semi-rigid panels that curve in a single direction along the midline of the face and sit on the face like a shield. Other masks utilize flexible multilayer constructions with silicone or similar materials to provide some degree of conformability to facial contours.

Existing phototherapy mask designs present various challenges related to facial conformity and treatment uniformity. When a mask fails to properly align with the three-dimensional contours of a user's face, bulging, gaps, and non-uniform spacing can occur between the mask surface and the skin. Due to the inverse square law governing light intensity, variations in the distance between LEDs and the target skin area result in differential optical power delivery across different facial regions. This can lead to inconsistent treatment outcomes, with some areas receiving more or less light energy than intended.

Conventional masks have employed various approaches to address facial fit, including adjustable straps, flexible materials, and cheek slits that allow the mask to bend in multiple directions. Some designs utilize coupling mechanisms to physically attach outer and inner layers together while allowing intermediate layers to slide relative to one another as the mask is bent. However, achieving a mask structure that remains thin, flexible, and capable of securely holding sensitive electronics while conforming closely to diverse facial anatomies remains challenging.

The construction of phototherapy masks also involves considerations related to protecting the LED assembly and associated circuitry from mechanical stress and environmental exposure. Various sealing and encapsulation techniques have been employed to house the electronic components within the mask structure. Additionally, the interface between connecting cables and the mask body can be subject to stress during handling and use, potentially affecting device durability.

Control systems for phototherapy masks have evolved to include various features for managing treatment parameters. Controllers may include power supplies, switches, and circuitry for regulating LED operation according to predefined treatment modes. Some systems incorporate user interfaces and displays for indicating device status and treatment information. The integration of intelligent features and energy-efficient display technologies in phototherapy mask controllers continues to be an area of development.

The present invention addresses the limitations of existing systems by introducing a flexible, multilayer ergonomic phototherapy mask system incorporating reinforced strap tension structures that eliminate bulging, a sealed thermoplastic polyurethane and polyurethane laminated flexible light panel assembly formed by ultrasonic welding, a strain-relieved cable interface, LED clearance regions that prevent compression during tightening, and functional openings for improved comfort. Additionally, the invention integrates intelligent controller functions such as GPS-based adaptive therapy and ultra-low-power E-Ink display technology. Collectively, these improvements overcome the shortcomings of conventional masks and deliver a thin, comfortable, durable, and highly uniform phototherapy experience tailored to the user's environment and anatomical features.

### OBJECTS OF THE INVENTION

Some of the objects of the invention are as follows:

An object of the present invention is to provide a phototherapy mask with a flexible mask body that conforms closely to the user’s facial contours, thereby improving comfort and ensuring effective light delivery.

Another object is to integrate eye openings that keep the wearer’s eyes unobstructed, while also incorporating secondary openings at the inner edges of those eye-opening areas to facilitate adjustable tensioning.

A further object is to include reinforced connecting positions on both sides of each eye-opening interior, which guide a tightening strap. When the strap is tensioned through these connecting positions, the adjacent sides of the mask body are drawn inward, pulling the mask snug against the face and minimizing bulging.

An additional object is to seal a thin flexible light between two flexible films, a translucent bottom film on the light-emitting side and a backlit top film on the rear side. The edges are joined by ultrasonic welding or adhesive bonding to form a sealed envelope encapsulating the panel.

Another object is to position the connecting positions outside the active LED region and at the seam of the sealed films, so that strap tension does not damage the LEDs or internal circuitry.

An object is to arrange the LED beads and circuit board with clearance zones so that the design ensures uniform irradiation and prevents stress on the LED elements during fit adjustment.

A further object is to incorporate additional cutouts for the nose and mouth that follow the nasal contour, and a mouth opening to accommodate breathing. These features adapt to facial anatomy and enhance user comfort without compromising treatment.

An object is to include a protective strain relief shell at the junction where the connecting cable meets the mask body. This shell resists tearing or delamination of the mask films under pulling forces, thereby reinforcing the cable, mask interface, and improving durability.

An additional object is to provide a user operable controller connected to the mask via a cable, the controller housing the control circuitry, power source, and a switch, which regulates power and control signals to flow to the mask’s flexible light panel.

A further object is to make the mask body from flexible polymer films such as thermoplastic polyurethane and polyurethane with optimized thickness 0.1–0.9 mm. These materials provide elasticity and comfort against the skin, while protecting the internal light panel.

A further object is to enable advanced controller features such as a built-in GPS receiver and location-based therapy profiles.

An additional object is to incorporate a low-power display, an electrophoretic E-Ink panel on the controller housing. This display can show therapy status mode, intensity, etc., with minimal power consumption, improving usability while preserving battery life.

Another object is to make the mask body lightweight and thin, reducing wearer fatigue and improving compliance during extended therapy sessions.

A further object is to achieve an overall phototherapy mask system in which the structural and functional elements work in harmony to deliver consistent, uniform illumination to the skin, while maximizing wearer comfort and device reliability.

### SUMMARY OF THE INVENTION

According to a first aspect of the present invention, a phototherapy mask is provided. The phototherapy mask comprising: a mask body being a flexible component, the mask body having two opening areas corresponding to eye regions of a user, each of the opening areas having an interior opening at one end away from the other opening area; one or more connecting positions disposed on both sides of each interior opening along a width direction thereof, the connecting positions configured to receive a strap to pull the mask body closer together along the width direction of the interior opening when the strap is connected, thereby reducing bulging between the mask body and a facial surface of the user; a flexible light panel disposed within the mask body, the flexible light panel comprising a substrate and a plurality of LED beads disposed on the substrate, wherein the substrate has a clearance area formed on one side of each interior opening along the width direction thereof and an overlapping area formed on an opposite side of each interior opening along the width direction, the overlapping area being configured to overlap the clearance area when two of the connecting positions are pulled together, the LED beads being located outside the clearance area; and a controller electrically connected to the mask body, the controller configured to control the operation of the mask body.

In one embodiment of the invention, the mask body further comprises a translucent bottom film and a backlit top film, the translucent bottom film and the backlit top film being flexible layers, the translucent bottom film being located on a light-emitting side of the flexible light panel, and the backlit top film being located on a backlit side of the flexible light panel.

In one embodiment of the invention, areas of the translucent bottom film and the backlit top film are both larger than an area of the flexible light panel, and edges of the translucent bottom film and the backlit top film are connected to seal the flexible light panel, the connecting positions being disposed on an outside of the flexible light panel and located at a connection between the translucent bottom film and the backlit top film.

In one embodiment of the invention, the translucent bottom film is a thermoplastic polyurethane layer, and the backlit top film is a polyurethane layer.

In one embodiment of the invention, thickness of the translucent bottom film is 0.1mm to 0.9mm.

In one embodiment of the invention, the edges of the translucent bottom film and the backlit top film are connected by at least one of ultrasonic welding, thermal welding, or adhesive bonding.

In one embodiment of the invention, the connecting positions are elongated through holes.

In one embodiment of the invention, the phototherapy mask further comprising a connecting cable electrically connecting the controller and the mask body, and a protective shell disposed at a connection point between the connecting cable and the mask body, the protective shell configured to prevent the mask body from being torn under force.

In one embodiment of the invention, the mask body is provided with a mouth opening corresponding to the mouth of the user, and a nose opening corresponding to the nose of the user, the mask body having a nose phototherapy area disposed within the nose opening, a top of the nose phototherapy area being connected to the mask body.

According to a second aspect of the present invention, a phototherapy mask system is provided. The phototherapy mask system comprising: a mask body comprising a translucent bottom film, a flexible light panel, and a backlit top film, wherein the translucent bottom film and the backlit top film are flexible layers, the translucent bottom film is located on a light-emitting side of the flexible light panel, and the backlit top film is located on a backlit side of the flexible light panel; wherein areas of the translucent bottom film and the backlit top film are larger than the area of the flexible light panel, and edges of the translucent bottom film and the backlit top film are connected to seal the flexible light panel; wherein the mask body has two opening areas corresponding to eye regions of a user, each opening area having an interior opening, and connecting positions disposed on both sides of each interior opening along a width direction thereof, the connecting positions configured to receive a strap to pull the mask body closer together along the width direction of the interior opening when the strap is connected; and a controller electrically connected to the mask body via a connecting cable, the controller comprising a housing, a control board, a battery, a switch button, and a GPS receiver, the GPS receiver configured to determine the location of the user and adjust therapy parameters based on the location.

In one embodiment of the invention, the controller is configured to adjust at least one of light intensity, therapy duration, or therapy timing based on at least one of a time zone or ambient sunlight conditions associated with the location.

In one embodiment of the invention, the flexible light panel comprises a substrate and a plurality of LED beads disposed on the substrate.

In one embodiment of the invention, the substrate has a clearance area formed on one side of the interior opening along the width direction thereof, and the substrate has an overlapping area formed on another side of the interior opening along the width direction thereof, the overlapping area being configured to overlap the clearance area when two of the connecting positions overlap, the LED beads being located outside the clearance area.

In one embodiment of the invention, the phototherapy mask system further comprising a protective shell disposed at a connection point between the connecting cable and the mask body, the protective shell configured to prevent the mask body from being torn under force.

In one embodiment of the invention, the edges of the translucent bottom film and the backlit top film are connected by at least one of ultrasonic welding, thermal welding, or adhesive bonding.

According to a third aspect of the present invention, a phototherapy mask is provided. The phototherapy mask comprising: a mask body being a flexible component having a translucent bottom film, a flexible light panel with a plurality of LED beads disposed on a substrate, and a backlit top film, wherein the translucent bottom film is a thermoplastic polyurethane layer, and the backlit top film is a polyurethane layer; wherein the mask body has two opening areas corresponding to eye regions of a user, each opening area having an interior opening at one end away from the other opening area; wherein the substrate has a clearance area formed on one side of each interior opening along a width direction thereof and an overlapping area formed on an opposite side of each interior opening along the width direction, the LED beads being located outside the clearance area, and the overlapping area being configured to overlap the clearance area when connecting positions on both sides of each interior opening are pulled together; and a controller electrically connected to the mask body, the controller comprising a housing, a control board, a battery, a switch button, and an electrophoretic display configured to display device status information, the electrophoretic display consuming power only when updating displayed content.

In one embodiment of the invention, the device status information comprises at least one of a therapy mode, an intensity level, an elapsed time, or a battery status.

In one embodiment of the invention, the electrophoretic display maximizes battery life during treatment by not drawing power in a static display mode.

In one embodiment of the invention, areas of the translucent bottom film and the backlit top film are larger than the area of the flexible light panel, and edges of the translucent bottom film and the backlit top film are connected to seal the flexible light panel.

In one embodiment of the invention, the edges of the translucent bottom film and the backlit top film are connected by at least one of ultrasonic welding, thermal welding, or adhesive bonding.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term "several" means more than one, unless otherwise specified.

In the context of the specification, the term “phototherapy mask system” refers to any device configured to emit therapeutic light for skin treatment, pain relief, or wellness applications.

In the context of the specification, the term “stimulation element” refers broadly to any component, module, or structure configured to apply a therapeutic or cosmetic stimulus to a user’s skin or tissue. Stimulation elements may include, but are not limited to, phototherapy elements, massage elements, microcurrent electrodes, ultrasonic transducers, heating elements, cooling elements, or combinations thereof.

In the context of the specification, the term “phototherapy element” encompasses any light-emitting device capable of emitting light of therapeutic wavelength(s), including but not limited to light-emitting diodes (LEDs), organic LEDs (OLEDs), laser diodes, or equivalent optical sources. The light may include ultraviolet, visible, near-infrared, or far-infrared spectra.

In the context of the specification, the term “microcurrent element” refers to any electrode or conductive structure configured to deliver a controlled electrical signal to the user’s skin. Such elements may include paired electrodes, conductive surfaces, or pads connected to a circuit board for generating microcurrent, galvanic current, or equivalent electrical therapy.

In the context of the specification, the term “housing” is intended to cover any casing, enclosure, or structural body that contains or supports components of the device. The housing may include a handle portion, head portion, or other segments, and may be made from polymeric, metallic, composite, or other suitable materials.

In the context of the specification, the terms “flexible light panel” or “light panel” refer to a portion of the device coupled to the housing and configured to emit light toward the skin. The flexible light panel may include one or more light-transmitting surfaces, optical lenses, or diffusers, and may also support electrodes or other stimulation elements.

In the context of the specification, the term “control interface” refers to any input or output mechanism enabling a user to operate the device. The control interface may include physical buttons, capacitive touch sensors, sliders, switches, or graphical displays, and may further include wireless control via a mobile application.

In the context of the specification, the term “circuit board” encompasses any printed circuit board (PCB), flexible circuit, or equivalent substrate that supports and electrically connects components of the device, including power supplies, control chips, drivers, or stimulation elements.

In the context of the specification, the term “user” or “subject” is intended to broadly cover humans, animals, or other recipients of the treatment, unless otherwise specifically limited.

In the context of the specification, the term "LED module" refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting

materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several ways depending on respective wavelength ranges, all of which are envisaged to be under the scope of this invention. For example, UV radiation can be categorized as Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm). Similarly, IR radiation may also be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm).

Unless otherwise stated, the term “light” as used in this specification encompasses electromagnetic radiation in the visible (380–780 nm) and infrared (780 nm–1000 nm) ranges, particularly red light (620–750 nm) and near-infrared (750–1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

The red light (approximately 630–660 nm) penetrates deeply into the scalp to stimulate blood circulation and enhance hair follicle activity, thus promoting hair growth and repair. Blue light (around 415–470 nm) exhibits antibacterial properties and is effective in treating scalp acne and reducing inflammation. Green light (approximately 520–540 nm) can help reduce pigmentation and soothe sensitive or irritated scalp tissue. Yellow light (around 580–600 nm) improves oxygen exchange in the cells and aids in detoxifying the scalp, while near-infrared light (800–850 nm) reaches deeper layers to accelerate healing and reduce pain and inflammation. Green light (approximately 520–540 nm) can help reduce pigmentation and soothe sensitive or irritated scalp tissue. Yellow light (around 580–600 nm) improves oxygen exchange in the cells and aids in detoxifying the scalp, while near-infrared light (800–850 nm) reaches deeper layers to accelerate healing and reduce pain.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 shows a phototherapy mask system, in accordance with an embodiment of the present invention.

FIG. 2 is an enlarged view of A in FIG. 1, in accordance with an embodiment of the present invention.

FIG. 3 shows an exploded view of the the phototherapy mask system, in accordance with an embodiment of the present invention.

FIG. 4 shows a perspective view of the controller, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

In one embodiment, the present invention provides a flexible phototherapy mask designed to closely conform to a user’s facial contours while delivering controlled light therapy comfortably and effectively. The mask includes a flexible mask body with two eye-region opening areas, each having an interior opening provided with connecting positions on both sides. A strap can be attached to these connecting positions to pull the mask body together in the width direction, reducing bulging and improving contact between the mask and the user’s face. Within the mask body, a flexible light panel is sealed between a translucent bottom film and a backlit top film, both of which extend beyond the light panel to protect and encapsulate it. The flexible light panel includes a substrate carrying multiple LED beads, with a clearance area and an overlapping area arranged on opposite sides of each interior opening so that, when the mask is tightened, the overlapping area covers the clearance area without placing LEDs in stressed regions. The mask is electrically connected to a controller that manages the operation of the LEDs and may include components such as a battery, control board, switch button, low-power electrophoretic display for status information, and optional GPS functionality to adjust therapy parameters based on the user’s location, thereby providing a smart, adaptive, and user-friendly phototherapy system.

In an embodiment, the mask body's multilayer construction provides a sealed light-emitting assembly. The mask includes a translucent bottom film on the inner side facing the skin, and a backlit top film on the outer side. Sandwiched between these films is a flexible light panel that carries a plurality of light-emitting diodes or equivalent light sources. Both the bottom and top films are sized larger than the flexible light panel. The peripheral edges are sealed together by at least one of ultrasonic welding or adhesive bonding to form an enclosed envelope around the flexible light panel and LEDs. This encapsulation protects the LEDs and circuitry from mechanical stress and environmental exposure, while keeping the overall profile of the mask very thin and pliable.

In an embodiment, the placement of LEDs on the flexible light panel is carefully arranged to maintain functionality when the mask is tensioned. On the flexible light panel, there is a designated clearance region along one side of each interior opening and an overlapping region along the opposite side. No LEDs are placed in the clearance region. When the strap at the connecting positions is pulled together, the overlapping portion of the flexible light panel is free to slide over the clearance region without compressing any LEDs. This design ensures that tightening the mask does not block light emission or impose bending stress on the light sources. The remaining LEDs positioned outside the clearance areas thus provide a controlled and uniform irradiation pattern across the treatment areas of the face.

In another embodiment, additional cutouts are provided in the mask body to improve user comfort and treatment coverage. A nose opening is included to accommodate the user's nose and house a dedicated nose phototherapy area at its upper portion to specifically treat the nasal bridge region. A mouth opening is provided to allow free breathing and to prevent coverage of the lips. These optional openings may be shaped to fit various facial anatomies and ensure that key treatment areas receive light while sensitive areas are exposed as needed.

In an embodiment, at the point where the electrical connecting cable attaches to the mask body, a protective shell for strain relief is provided. The protective shell reinforces the interface between the mask’s films and the connecting cable, distributing stress and preventing tearing or delamination of the film layers when the mask is handled or when the strap applies force. By strengthening this junction, the protective shell helps maintain the structural integrity of the sealed flexible light panel and prolongs the lifetime of the device under repeated use.

In an embodiment, the translucent bottom film and backlit top film are composed of flexible polymer materials, specifically thermoplastic polyurethane and polyurethane. The translucent bottom film is a thin thermoplastic polyurethane layer, approximately 0.1 mm to 0.9 mm thick, that offers excellent flexibility, optical clarity, and durability. The backlit top film is a softer polyurethane material that provides a pleasant tactile feel and elasticity. Together, these layers form a comfortable, breathable interface against the skin while firmly holding the LED assembly on the flexible light panel between them. The translucent bottom film and backlit top film are joined by at least one of ultrasonic welding, thermal welding, or adhesive lamination around the edges to create a continuous seal that resists moisture and wear.

The use of polyurethane material for constructing the mask body provides several technical, user-centric, and manufacturing advantages that make it particularly suitable for phototherapy devices. With respect to comfort and ergonomics, the soft and flexible nature of polyurethane allows the mask body to conform closely to facial contours, ensuring uniform contact with the skin. The material properties may reduce pressure points, enabling extended wear during light therapy sessions without discomfort. Polyurethane provides biocompatibility and skin safety advantages. Medical-grade polyurethane may be non-toxic, hypoallergenic, and skin-safe, minimizing risks of irritation or allergic reactions. The material may be suitable for direct and prolonged skin contact, which is an important requirement for therapeutic facial devices. Polyurethane offers optical and thermal compatibility benefits. Polyurethane may be engineered to be light-diffusive or light-transmissive, helping distribute therapeutic light evenly across treatment areas. The material may exhibit low thermal conductivity, providing insulation and preventing heat buildup from the LED beads or other light-emitting elements on the flexible light panel. The material may offer excellent tear resistance, abrasion resistance, and fatigue resistance, ensuring long service life despite repeated flexing of the mask body. Polyurethane may maintain its shape and mechanical properties under routine cleaning and daily use. The low density of polyurethane reduces overall mask weight, enhancing user compliance during therapy sessions and reducing wearer fatigue. The material may be resistant to sweat, oils, and moisture, which is beneficial for facial applications. Polyurethane may tolerate common sanitizing agents, supporting hygiene and reuse of the phototherapy mask. The material may be molded into complex geometries, allowing integration of LED arrays, wiring channels, ventilation features, and fastening elements. Polyurethane may be compatible with over-molding processes, enabling seamless encapsulation of electronic components for improved safety and aesthetics. The material may offer shock and vibration absorption, protecting delicate light-emitting components such as the LED beads during handling or transport of the phototherapy mask. The material may be readily available and may be cut easily into any shape using molds and dies, supporting scalable mass production of the mask body.

In an embodiment, the flexible light panel is precisely aligned between the translucent bottom film and the backlit top film. The three-layer stack is sealed at the perimeter by ultrasonic welding to form a unified, laminated structure. Ultrasonic welding securely fixes the flexible light panel in place and prevents it from shifting or detaching when the mask flexes. The sealing also creates a reinforced band at the border of the active area, which is where the connecting positions for the straps are located. The mechanical forces from tightening the strap are borne by the robust film seam rather than the internal electronics.

In an embodiment, the connecting positions are implemented as through holes or slots formed in the joint film region on either side of each interior opening. These connecting positions are elongated to accept the strap and allow the two sides to overlap when tightened. They are reinforced by the welded film edges so that they resist tearing. The connecting positions lie outside the LED-covered area of the panel, ensuring that no light is blocked by the strap and no LEDs are located in these holes or in the adjacent overlap zones. This arrangement further ensures safe and effective use of the strap mechanism.

In an embodiment, an extended cut-out area is provided in the mask body above the eyes, encompassing part of the forehead or temple region. This extended opening allows the mask to flex more freely along the brow line, accommodating variations in forehead slope or eyebrow position without wrinkling. The mask employs a dual-strap tension system, one strap engaging the connecting positions near the upper extended opening, tightening the top portion of the mask, and a second strap engaging positions near the lower face or chin and cheek area. By adjusting the tension of each strap independently, the user can achieve even closer contact across different facial zones. The two-axis tightening capability effectively eliminates the bulging effect over the entire face and adapts the mask to complex three-dimensional contours.

In an embodiment, the phototherapy mask system operates under the control of a separate controller unit. The controller contains a housing in which a control board and a power source, such as a rechargeable battery, are installed. A switch on the controller allows the user to activate or deactivate the LED illumination. Upon activation, the controller delivers regulated electrical power and control signals through the connecting cable to the flexible light panel inside the mask. The LEDs emit therapeutic light with specific wavelengths or color combinations for the predetermined session. Deactivating the switch cuts power to the LEDs, ending the treatment. The controller is designed for handheld and wearable use, and alternatively, it may be attached to the mask for an integrated form factor.

In some embodiments, the controller is further enhanced with intelligent features. The controller includes a global positioning system (GPS) receiver and programming connected to the controller circuit board that accesses a database of therapy parameters tailored to different geographic and environmental conditions. By determining the user's current location and local time zone, the controller can automatically adjust and suggest therapy settings. The location-based adjustments compensate for variations in ambient sunlight, ultraviolet exposure, or user circadian rhythm, ensuring consistent treatment efficacy regardless of where the user is located. The controller's firmware allows a special adaptive mode that dynamically sets the LED output based on the GPS-acquired data and the optimum therapy parameters for a particular duration and intensity.

In an embodiment, the controller incorporates a low-power electronic display to inform the user of the device's overall status. An electrophoretic display, also known as digital ink or E-Ink display, is employed, consuming power only when updating its content. Such a screen can show the selected phototherapy mode, current intensity level, elapsed time, battery status, and other relevant information with high visibility under various lighting conditions. Because the display does not draw power in static mode, it helps maximize battery life during treatment. The display is embedded under a protective transparent window in the controller housing, ensuring it remains visible and protected during handling.

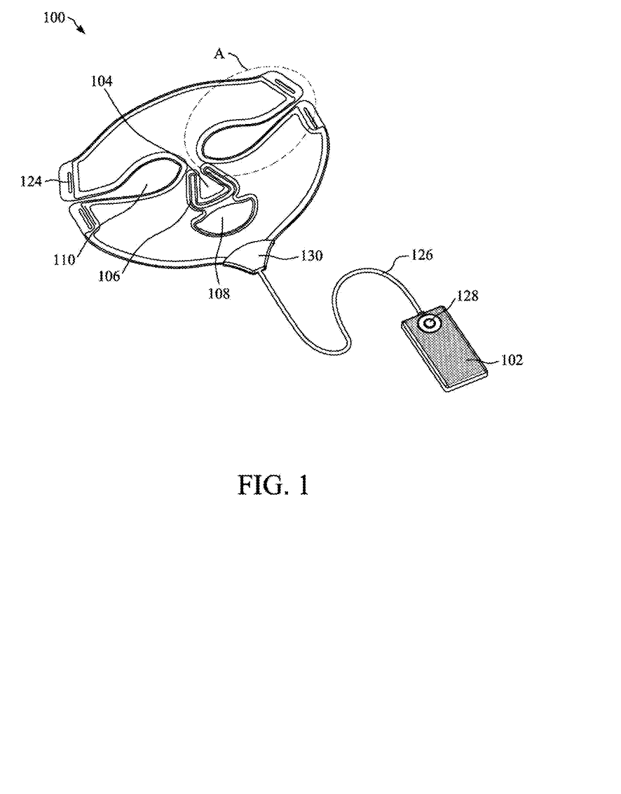

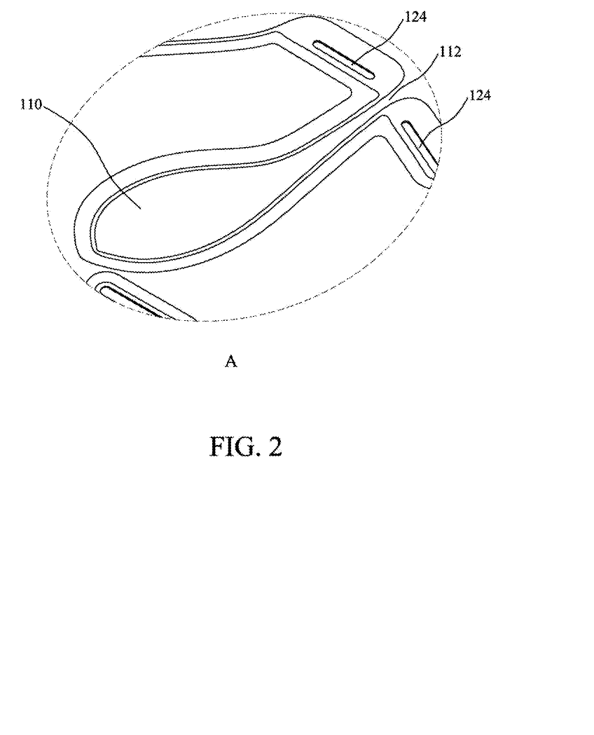

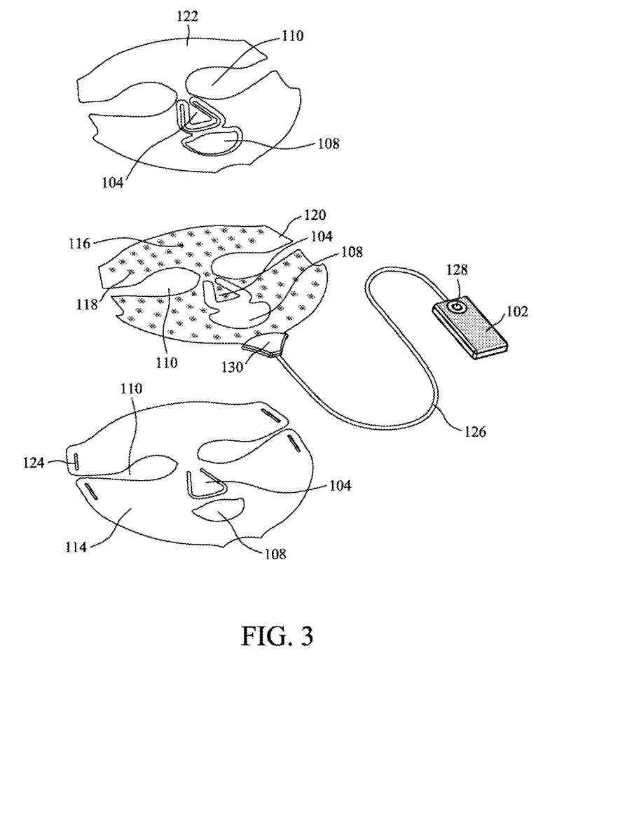

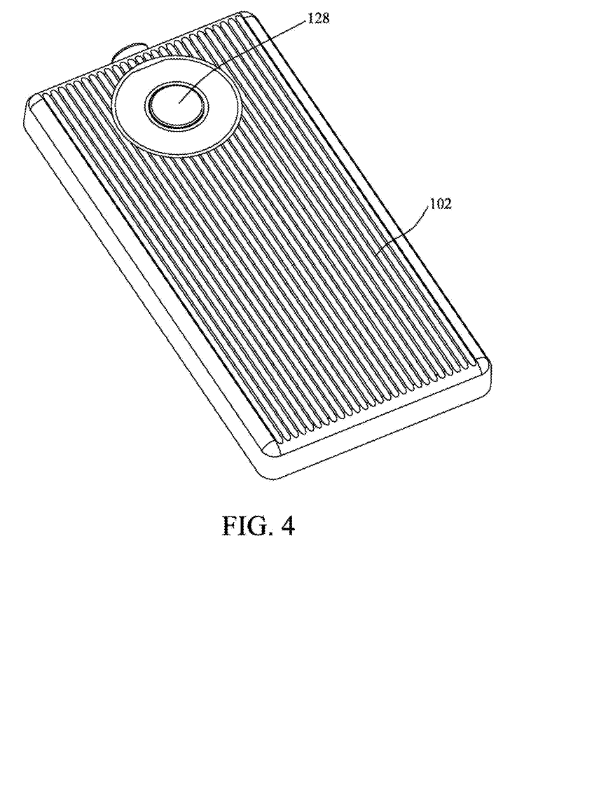

Embodiments of the present invention will now be described with reference to FIGS. 1 to 4.

Referring to FIGS. 1 to 4, the present invention proposes a phototherapy mask including a mask body 100 and a controller 102. The mask body 100 is a flexible component configured to conform to the contours of a user's face, thereby improving comfort and treatment efficacy. The mask body 100 is provided with two opening areas 110 corresponding to the user's eyes. An interior opening 112 is formed at one end of each opening area 110 away from the other opening area 110 so that the eyes remain unobstructed during therapy.

The mask body 100 further comprises one or more connecting positions 124 arranged on both sides of each interior opening 112 along the width direction of the opening. The one or more connecting positions 124 are configured to receive and guide a strap. When a strap is routed through the one or more connecting positions 124 and tensioned, the two sides of the mask body 100 adjacent to the interior opening 112 are drawn closer together along the width direction. This targeted tensioning causes the mask body 100 to seat tightly against the facial surface, substantially reducing bulging and height differential between the mask body 100 and the user's skin, thereby eliminating the discomfort.

The controller 102 is electrically connected to the mask body 100 via a connecting cable 126. The controller 102 houses a control board, a power source, and a user-operable switch button 128 that is electrically connected to the flexible light panel embedded in the mask body 100. Activation of the switch button 128 causes the controller 102 to supply power and control commands for a flexible light panel 116, thereby energizing LED beads 118 and initiating phototherapy. Deactivation of the switch button 128 ceases illumination and terminates the therapeutic session. The controller 102 may alternatively be mounted directly on or removably coupled to the mask body 100.

In preferred embodiments, the mask body 100 includes a translucent bottom film 114, the flexible light panel 116, and a backlit top film 122. Both the translucent bottom film 114 and the backlit top film 122 are flexible layers selected and configured to promote conformability while protecting and optically coupling with the flexible light panel 116. The areas of the translucent bottom film 114 and the backlit top film 122 are shaped to be larger than the active region of the flexible light panel 116, and their edges are sealed together to form a sealed envelope encapsulating the flexible light panel 116. The one or more connecting positions 124 are located outside the active region of the flexible light panel 116, at the interface of the translucent bottom film 114 and the backlit top film 122, to prevent damage to the illumination elements when the strap is tensioned.

In some embodiments, the flexible light panel 116 carries the plurality of LED beads 118 arranged to provide a controlled and uniform irradiation profile across the therapy areas. The flexible light panel 116 includes a clearance area 120 on one side of an interior opening 112 and an overlapping area on the opposite side. The overlapping area is configured to overlap the clearance area 120 when adjacent one or more connecting positions 124 are drawn together, and the LED beads 118 are positioned outside the clearance area 120 to avoid occlusion when the mask body 100 is folded or overlapped for fit adjustment.

In some embodiments, the LED beads 118 on the flexible light panel 116 may emit therapeutic light at specific wavelengths selected for photobiomodulation and skin treatment applications. The LED beads 118 may include red light-emitting diodes operating in the wavelength range of approximately 620-660 nm, which may promote collagen production, enhance blood circulation, and support skin rejuvenation. The LED beads 118 may also include near-infrared light-emitting diodes operating in the wavelength range of approximately 810-850 nm, which may penetrate deeper into tissue layers to promote cellular repair and reduce inflammation. In some embodiments, the LED beads 118 may include blue light-emitting diodes operating in the wavelength range of approximately 415-470 nm, which may provide antibacterial effects for acne treatment and skin clarification. The flexible light panel 116 may incorporate combinations of LED beads 118 emitting different wavelengths to provide multispectral therapy, and the controller 102 may be configured to selectively activate different wavelength groups according to the desired treatment protocol.

In some embodiments, to facilitate comfort and precise facial conformity, the mask body 100 is provided with additional cutouts corresponding to a nose phototherapy area 104, a nose opening 106, and a mouth opening 108. The nose phototherapy area 104 is arranged within the nose opening 106, and the top of the nose phototherapy area 104 is integrated with the mask body 100 so that the device follows the nasal contour closely. The optional mouth opening 108 accommodates variations in facial geometry and user breathing requirements.

A protective shell 130 is provided at the junction between the connecting cable 126 and the mask body 100 to prevent tearing of the translucent bottom film 114 and the backlit top film 122 under tensile loading. The protective shell 130 reinforces the mechanical connection and improves the durability of the interface against repeated handling and strap-induced tension.

In some embodiments, through the provision of strategic opening areas 110, dedicated one or more connecting positions 124, and a sealed flexible illumination assembly of translucent bottom film 114, flexible light panel 116, backlit top film 122, the invention achieves a highly conformable phototherapy mask system that minimizes bulging, increases wearer comfort, and enables consistent, effective phototherapeutic delivery under user controlled operation via the controller 102.

In some embodiments, the controller 102 is configured to control the operational state of the mask body 100. The connecting cable 126 is used to electrically couple the controller 102 to the mask body 100, thereby enabling transmission of power and control signals between these components.

In another embodiment, a plurality of interior openings 112 are formed within the perforated opening areas 110 of the mask body 100, and one or more connecting positions 124 are arranged on both lateral sides of each respective interior opening 112. During use, a strap is routed through the one or more connecting positions 124, which allows the user to apply targeted tension to draw the mask body 100 toward the face. This structural arrangement ensures that the mask body 100 adheres securely to the facial surface, thereby preventing slippage, misalignment, or sagging during phototherapy.

In some embodiments, the raised contour of the mask body 100 is designed to generally conform to the anatomical curvature of the user's face, effectively mitigating the tendency of conventional phototherapy masks to bulge outward and cause discomfort. This configuration improves both comfort and optical efficiency by maintaining a minimal air gap between the light-emitting structures and the user's skin. Furthermore, the structural simplicity of the mask body 100 allows for reduced manufacturing cost, straightforward processing, and simplified assembly methods.

In some embodiments, the mask body 100 is fabricated as a flexible component to enhance its conformance characteristics. The mask body 100 includes two opening areas 110 corresponding to the user's eyes, and each opening area 110 includes an interior opening 112 positioned at the end farthest from the other opening area 110. The one or more connecting positions 124 are positioned along the width direction of each interior opening 112. When a strap is engaged with the one or more connecting positions 124, lateral tension is applied, which draws the mask body 100 inward along the width direction of the interior opening 112. This results in a more intimate fit against the user's face and prevents wrinkling or folding of the mask during application.

In some embodiments, the mask body 100 comprises the translucent bottom film 114, the flexible light panel 116, and the backlit top film 122. The translucent bottom film 114 and the backlit top film 122 are flexible layers that encapsulate and protect the illumination assembly. The translucent bottom film 114 is disposed on the light-emitting side of the flexible light panel 116, whereas the backlit top film 122 is disposed on the reverse, non-emitting side of the flexible light panel 116. Such an arrangement enables effective diffusion and transmission of light generated by the LED beads 118, while ensuring that the internal circuitry remains shielded from external mechanical stress.

In some embodiments, the flexible light panel 116 includes the designated clearance area 120 corresponding to one side of an interior opening 112 and, on the opposite side, an overlapping region configured to overlap the clearance area 120 when the one or more connecting positions 124 are tightened. The LED beads 118 are intentionally located outside the clearance area 120 so that they are not obstructed or mechanically stressed when the mask is tensioned for fitting. This anticipatory design ensures uniform illumination distribution, enhanced operational stability, and prolonged product life.

In an embodiment, cutouts may optionally be provided in the mask body 100, such as the mouth opening 108, the nose opening 106, and the associated nose phototherapy area 104, depending on treatment requirements and user comfort preferences.

Through the combined use of strategically located interior openings 112, reinforced one or more connecting positions 124, and an integrated multilayer optical structure composed of the translucent bottom film 114, flexible light panel 116, and backlit top film 122, the present phototherapy mask system delivers improved facial conformity, enhanced user comfort, and stable, uniform light output. These design optimizations contribute to a more reliable and commercially efficient phototherapy system.

In certain embodiments, the translucent bottom film 114 and the backlit top film 122 are dimensioned such that their respective surface areas are each greater than the surface area of the flexible light panel 116. The peripheral edges of the translucent bottom film 114 and the backlit top film 122 are joined together by at least one of thermal welding, ultrasonic welding, adhesive bonding, or any equivalent sealing method to encapsulate and hermetically secure the flexible light panel 116 within a protected interior region. This multilayer construction improves environmental resistance, limits mechanical strain on the LED beads 118, and enhances the overall durability of the mask body 100.

In some embodiments, the one or more connecting positions 124 are positioned outside the perimeter of the flexible light panel 116, specifically at or near the seam where the translucent bottom film 114 and the backlit top film 122 are joined. This placement ensures that tension forces applied through the strap are transferred directly to the strengthened boundary region of the film layers, thereby preventing tearing or delamination of the optical structures.

In some embodiments, when the mask body 100 is in use, the opening areas 110 each include an interior opening 112 that provides structural flexibility. As the strap passes through the one or more connecting positions 124 at both sides of each interior opening 112, the lateral pulling forces reduce the bulging effect typically observed in flexible phototherapy masks. The resulting height difference between the central region and the peripheral region of the mask body 100 becomes minimal, enabling a more uniform surface contact between the phototherapy interface and the user's skin. By reducing bulging, the interior openings 112 effectively improve comfort, enhance phototherapy efficiency, and maintain stable adherence during use.

In some embodiments, the mask body 100 may include four connecting positions 124 arranged symmetrically. This configuration provides multi-directional pulling capability, allowing the mask to be tensioned evenly along its width and height, further reducing the possibility of deformation or bulging and enhancing the conformity of the mask to diverse facial structures.

In some embodiments, the flexible light panel 116 comprises a substrate on which multiple LED beads 118 are mounted. The substrate includes a designated clearance area 120 formed along one side of each interior opening 112 in the width direction. Opposite to the clearance area 120, an overlapping region is provided along the other side of the interior opening 112. During use, when the one or more connecting positions 124 on opposite sides of the interior opening 112 are pulled together, causing partial overlapping of the structure, the overlapping region is designed to overlay the clearance area 120. Importantly, the LED beads 118 are intentionally positioned outside the clearance area 120, ensuring that no active light-emitting components reside in regions subjected to compression, bending, or overlapping. This arrangement significantly improves electrical reliability, reduces stress-induced fatigue, and ensures consistent optical output during repeated tightening cycles.

In some embodiments, when the mask body 100 is in use, the user may activate the device by pressing the switch button 128, which communicates with the controller 102. Upon activation, the controller 102 supplies regulated power to the flexible light panel 116 through the connecting cable 126, causing the LED beads 118 to emit therapeutic light. This emission enables the phototherapy function of the mask body 100. On deactivating the system via the switch button 128, the controller 102 interrupts power delivery to the flexible light panel 116, thereby extinguishing the LED beads 118 and terminating the phototherapy cycle.

The coordinated interaction of the translucent bottom film 114, flexible light panel 116, backlit top film 122, opening areas 110, and strategically placed one or more connecting positions 124 provides a technically robust and user-comfortable phototherapy mask system architecture. These structural and functional enhancements address the shortcomings of conventional masks by minimizing bulging, improving adhesion, extending device longevity, and delivering more uniform and effective phototherapeutic illumination.

In some embodiments, the flexible light panel 116 includes the designated clearance area 120 positioned at the region corresponding to the interior opening 112 of the opening area 110. Within this clearance area 120, no LED beads 118 are provided. The absence of light-emitting components in this region allows the mask body 100 to be partially folded or overlapped when worn without imposing mechanical stress on the illumination circuitry. The LED beads 118 may be replaced with an equivalent light-emitting electronic element, such as a micro-LED module, OLED element, or alternative semiconductor-based emitter.

In an embodiment, a through-hole is formed in the central region of the flexible light panel 116, enabling light from the LED beads 118 or other illumination sources to pass through the corresponding aperture. The translucent bottom film 114 is welded or bonded to the backlit top film 122 to provide additional structural reinforcement and to further prevent bulging of the translucent bottom film 114 during use.

In an embodiment, both the translucent bottom film 114 and the backlit top film 122 are formed as a single integrated film, thereby simplifying manufacturing and improving flexibility. The thickness of the translucent bottom film 114 is selected from 0.1 mm to 0.9 mm. More specifically, the translucent bottom film 114 and the backlit top film 122 layers are manufactured with thicknesses of 0.3 mm, 0.5 mm, or 0.6 mm, depending on the degree of flexibility, elasticity, and mechanical support desired. By constructing the translucent bottom film 114 from thermoplastic polyurethane material and the backlit top film 122 from polyurethane material, the overall mask body 100 becomes highly flexible, lightweight, and capable of conforming closely to the contours of the user's face, thereby improving comfort and enhancing phototherapeutic efficacy.

In some embodiments, the flexible light panel 116 is positioned between the translucent bottom film 114 and the backlit top film 122. The layer interfaces between the translucent bottom film 114 and the flexible light panel 116, as well as between the flexible light panel 116 and the backlit top film 122, are joined through thermal welding, ultrasonic bonding, or adhesive lamination. All three components, the translucent bottom film 114, the flexible light panel 116, and the backlit top film 122, may be sealed together through a continuous adhesive layer, thereby forming a unified multilayer structure.

In an embodiment, during manufacturing of the mask body 100, layers of thermoplastic polyurethane material may first be cut or die-shaped to form the translucent bottom film 114 and the backlit top film 122. The flexible light panel 116 is then placed or laminated between these two layers, and the peripheral regions of the translucent bottom film 114 and backlit top film 122 are joined in combination, or at least one of ultrasonic welding, hot-bar welding, or adhesive bonding, to encapsulate and seal the flexible light panel 116.

In some embodiments, the manufacturing process for the mask body 100 may include the following steps. First, the thermoplastic polyurethane material and polyurethane material are cut or die-shaped to form the translucent bottom film 114 and the backlit top film 122, respectively, with appropriate openings corresponding to the opening areas 110, nose opening 106, and mouth opening 108. Second, the flexible light panel 116 is prepared by mounting or soldering the LED beads 118 onto the substrate, with the clearance areas 120 left free of electronic components. Third, the flexible light panel 116 is positioned between the translucent bottom film 114 and the backlit top film 122, with the LED beads 118 facing the translucent bottom film 114. Fourth, the peripheral edges of the translucent bottom film 114 and the backlit top film 122 are aligned and sealed together using ultrasonic welding at a frequency range of approximately 20-40 kHz, or alternatively using thermal welding at appropriate temperatures for the polymer materials, or adhesive bonding. Fifth, the one or more connecting positions 124 are formed as through-holes in the sealed edge regions on both sides of each interior opening 112. Sixth, the connecting cable 126 is attached to the flexible light panel 116, and the protective shell 130 is secured at the junction between the connecting cable 126 and the mask body 100 to provide strain relief.

In some embodiments, each of the one or more connecting positions 124 is formed as a through-hole that facilitates the insertion of a strap. The through-hole may be elongated to create a slot-like geometry, enabling better mechanical engagement with the strap and allowing the two through-holes located on opposite sides of the interior opening 112 to be overlapped when tensioned. Overlapping the through-holes enhances the conforming fit of the mask body 100 to the user's face while reducing bulging. In alternative embodiments, the through-hole at each of the one or more connecting positions 124 may be circular, arc-shaped, or otherwise contoured to accommodate different strap configurations or manufacturing preferences.

In further embodiments, the mask body 100 is provided with a mouth opening 108 corresponding to the user's mouth and a nose opening 106 corresponding to the nose. The dedicated nose phototherapy area 104 is positioned within or adjacent to the nose opening 106, enabling targeted illumination for nasal skin regions. The upper region of the nose phototherapy area 104 may be integrally connected to the mask body 100, ensuring improved structural support and enabling the mask body 100 to conform more effectively to the nasal and mid-facial contours of the user. The mouth opening 108 and nose opening 106 are configured in various shapes or geometries to adapt to different facial anatomies. The nose opening 106 may be entirely open or may be formed as a semi-open structure, allowing selective coverage around the nasal region while maintaining breathability and user comfort.

In some embodiments, the described structural and material selections including the thermoplastic polyurethane based film layers, reinforced one or more connecting positions 124, selectively positioned LED beads 118, and strategically designed clearance area 120 collectively enhance the flexibility, stability, and light delivery performance of the mask body 100, providing a phototherapy mask system that is comfortable, durable, and optimized for effective cosmetic and therapeutic illumination.

In some embodiments, the controller 102 is equipped with the switch button 128, which is electrically coupled to the mask body 100 through the connecting cable 126. At the junction where the connecting cable 126 interfaces with the mask body 100, the protective shell 130 is provided. The protective shell 130 functions to resist mechanical tearing, reduce strain concentration, and increase long-term durability of the mask during repeated donning, doffing, and cable manipulation.

In one embodiment, the controller 102 comprises an external housing, an internal control board, a power supply such as a rechargeable battery, and the previously mentioned switch button 128. The control board is securely mounted within the housing and electrically connected to the internal battery. The switch button 128 is integrated directly onto or operably connected to the circuitry of the control board, enabling the user to activate, deactivate, or adjust the output of the phototherapy system with minimal actuation force. The ergonomic placement of the switch button 128 ensures intuitive operation in handheld or wearable conditions.

In an embodiment, the phototherapy mask may comprise one or more stimulation components selected from a phototherapy component, a microcurrent component, a magneto-therapy component, a Peltier component, a heating and, a cooling component, and an ultrasonic wave therapy component. Each stimulation element may operate independently or in combination with the others to provide a multi-modal treatment experience.

In an embodiment, the stimulation component can be the microcurrent component configured to provide microcurrent stimulation therapy. The microcurrent component may include one or more electrodes disposed on the surface of the housing, such as on the end face or connecting surface, and electrically connected to the internal circuit board. The circuit board is configured to generate controlled low-level electrical pulses that are delivered through the electrodes to the user’s skin. Such microcurrent stimulation aids in promoting circulation, improving cellular energy (ATP) production, and enhancing skin tone and elasticity. The microcurrent component may operate independently or concurrently with other stimulation elements such as the phototherapy, thermal, or ultrasonic modules to produce synergistic cosmetic and therapeutic effects.

In an embodiment, the stimulation component can be the heating and cooling component configured to provide controlled thermal therapy. The heating and cooling component may include a Peltier module, a resistive heating plate, or a thermoelectric component disposed beneath or adjacent to the light-transmitting plate. The thermal component is thermally coupled to the surface of the housing so that heat or coolness is effectively transferred to the user’s skin. The control circuit regulates the direction and magnitude of current through the Peltier component to alternately produce heating or cooling effects.

In an embodiment, the stimulation component can be the magneto-therapy component that may include one or more electromagnetic coils or permanent magnets configured to generate a pulsed or static magnetic field to promote blood circulation and cellular metabolism.

The stimulation component can be the ultrasonic wave therapy component that may include one or more piezoelectric transducers adapted to emit ultrasonic vibrations in the range of 0.8-3 MHz to stimulate tissue regeneration, enhance transdermal absorption of skincare products, and relieve muscular tension.

In an embodiment, the phototherapy component provides optical stimulation using specific wavelengths of light, while the microcurrent component delivers controlled low-level electric currents through electrodes on the device surface. The heating and cooling components, including the thermoelectric (Peltier) component, regulate the surface temperature to deliver thermal therapy for soothing or tightening skin. All these components may be controlled individually or simultaneously through the circuit board and user interface, allowing the user to select desired therapy modes depending on treatment needs.

In some embodiments, the phototherapy mask further includes the connecting cable 126, which establishes the electrical communication between the control board housed in the controller 102 and the illumination components embedded in the mask body 100. To minimize cable fatigue and inhibit damage caused by repeated bending, pulling, or torsional stress, the protective shell 130 is arranged at the one or more connecting positions 124 between the connecting cable 126 and the mask body 100. The incorporation of the protective shell 130 not only prevents tearing but also reinforces the seal where the translucent bottom film 114 and the backlit top film 122 interface, thereby maintaining the integrity of the internal illumination structure.

In another embodiment, the addition of the protective shell 130 at the one or more connecting positions 124 increases the overall structural robustness of the mask body 100 and mitigates the risk of delamination or separation between the translucent bottom film 114 and the backlit top film 122 when subjected to pulling or stretching forces. This protective measure ensures the internal flexible light panel 116 remains securely enclosed and that the phototherapy device retains its designed optical performance without risk of light leakage or panel deformation.

In some embodiments, the mask body 100 includes backlit top film 122 and translucent bottom film 114 that jointly form an encapsulating structure for the flexible light panel 116. The backlit top film 122 of the mask body 100 is made of a polyurethane material, while the translucent bottom film 114 is made of a thermoplastic polyurethane material. The polyurethane material used on the backlit top film 122 provides enhanced softness, skin-friendliness, and long-term wear comfort during phototherapy application. The thermoplastic polyurethane material used on the translucent bottom film 114 provides improved mechanical strength, tear-resistance, and overall structural stability for the mask body 100.

The combination of polyurethane material and thermoplastic polyurethane material increases flexibility while maintaining durability. The thermoplastic polyurethane surface acts as the primary skin-contacting layer, reducing irritation and improving breathability. The polyurethane layer on the opposite side protects the internal flexible light panel 116 from external mechanical stress and deformation. As the polyurethane and thermoplastic polyurethane layers together surround the flexible light panel 116, they also contribute to improved environmental sealing and reduce the risk of moisture infiltration that may affect the functioning of the light-emitting components, including LED beads 118.

In this embodiment, the one or more connecting positions 124, interior openings 112, opening areas 110, and other structural functional features remain consistent with the described embodiments. The use of polyurethane and thermoplastic polyurethane allows these structural portions to deform appropriately when the strap is inserted and pulled through the one or more connecting positions 124, thereby enhancing the ergonomic fit of the mask body 100 and further reducing bulging during use.

In another embodiment, the mask body 100 includes a multilayer stacked structure in which the flexible light panel 116 is sandwiched between a polyurethane layer and a thermoplastic polyurethane layer. The flexible light panel 116 layer supports the LED beads 118 or other light-emitting electronic components used to perform phototherapy. The thermoplastic polyurethane layer forms the translucent bottom film 114 surface of the mask body 100, and the polyurethane layer forms the backlit top film 122 surface.

In another embodiment, the flexible light panel 116 is placed between the polyurethane backlit top film 122 and the thermoplastic polyurethane translucent bottom film 114. The three layers are then bonded together by ultrasonic welding or, alternatively, by glue-based adhesion. Ultrasonic welding allows quick and secure joining without introducing excessive heat that may damage the flexible light panel 116 or LED beads 118. Glue adhesion may be used as another option to achieve reliable bonding, depending on manufacturing requirements.

In another embodiment, the polyurethane-flexible light panel 116 and thermoplastic polyurethane laminate forms a sealed composite structure in which the flexible light panel 116 is fully embedded. This structure provides multiple advantages, including improved mechanical protection of the flexible light panel 116, better waterproofing, and enhanced comfort when the mask is worn, adapting to the body contours. The embedded flexible light panel 116 ensures stable electrical performance while maintaining the overall thinness and flexibility of the mask body 100. In addition, with the flexible light panel 116 sandwiched and fixed between the polyurethane material sheet and the thermoplastic polyurethane material sheet layers, the risk of detachment or misalignment is significantly reduced even when the mask body 100 is repeatedly bent or pulled during use.

In another embodiment, the one or more connecting positions 124, interior openings 112, controller 102 interface, and light-transmitting regions of the mask body 100 remain compatible with this laminated structure. The sandwich arrangement also ensures that areas corresponding to clearance area 120 around opening regions can be fabricated without LED beads 118 or electronic components, preventing blockage of the light path and facilitating smooth folding or overlapping around the one or more connecting positions 124.

In an embodiment, the mask body 100 is a multilayer flexible structure composed of a polyurethane backlit top film 122, a thermoplastic polyurethane translucent bottom film 114, and the flexible light panel 116 disposed between the polyurethane backlit top film 122 and the thermoplastic polyurethane translucent bottom film 114. The polyurethane first surface serves as the outer surface of the mask body 100 and provides softness, skin friendliness, and elastic recovery, thereby improving the comfort of wearing the mask for long durations. The thermoplastic polyurethane second surface forms the inner light-emitting side and exhibits excellent optical transparency, mechanical flexibility, and heat resistance, ensuring stable light output of the flexible light panel 116.

In another embodiment, the flexible light panel 116 is arranged between the polyurethane and thermoplastic polyurethane layers. The flexible light panel 116 layer carries a plurality of LED beads 118 or other light-emitting components. In order to ensure mechanical reliability and to avoid damage when the mask is bent or pressed against the user's facial contour, the flexible light panel 116 may be designed with a reduced-thickness region corresponding to the opening area 110 or an extended cut-out of the mask body 100. The flexible light panel 116 may further include the clearance area 120 in locations where the mask folds or overlaps during tightening.

In another embodiment, the bonding between the polyurethane layer and the thermoplastic polyurethane layer may be performed using ultrasonic welding, thermal welding, or adhesive bonding, ensuring a sealed, laminated structure that encapsulates the flexible light panel 116. Ultrasonic welding can provide a strong, continuous seam around the edges, preventing the flexible light panel 116 from shifting and ensuring liquid resistance and structural integrity. Adhesive bonding may also be used to reinforce the bonding area and improve cushioning between the flexible light panel 116 and the outer PU layer.

In another embodiment, the polyurethane-thermoplasticurethane PCB sandwich structure provides enhanced durability, a thin profile, and superior flexibility. When combined with the interior openings 112 and one or more connecting positions 124 described in the earlier embodiments, this multilayer construction enables the mask body 100 to maintain close contact with the user's face while resisting tearing, delamination, or bulging during tightening. The flexible light panel 116 inside the polyurethane/thermoplastic polyurethane structure allows effective light therapy with uniform luminance while improving the overall comfort and safety of the phototherapy mask.

In an embodiment, the mask body 100 is configured with the extended opening area 110 in the facial region corresponding to the user's eyes and upper-forehead area. This extended opening area 110 enlarges the conventional eye-opening zone and allows the mask body 100 to flex more naturally in the region above the eyes, thereby accommodating variations in forehead curvature, eyebrow height, and eye-socket depth among different users. The extended cut-out reduces concentrated bending stress around the eye aperture and facilitates smoother stretching of the mask body 100 during tightening. The mask body 100 conforms more precisely to the contours of the user's forehead and orbital regions without causing wrinkling or bulging.

In some embodiments, cooperating with the extended opening area 110, a dual-strap tension system is provided. Specifically, separate connecting positions 124 are provided beside the interior opening 112, and another set of connecting positions 124 is provided beside the facial region.

In some embodiments, a first strap passes through the one or more connecting positions 124 situated near the extended opening area 110 along its width direction region to apply a targeted pulling force to the upper portion of the mask body 100, including the forehead, temple, and upper-cheek areas. This ensures that the upper mask portion fits closely to the curved surface above the eyes.

In some embodiments, a second strap passes through the one or more connecting positions 124 located near the lower face area and exerts controlled tension on the mid-cheek, side-cheek, and chin regions. By independently adjusting the first and second straps, the mask body 100 can be tightened along two separate tightening axes, one corresponding to the forehead-eye region and the other corresponding to the facial region. This configuration allows the mask body 100 to adapt to complex three-dimensional facial contours, eliminating the bulging effect commonly found in conventional single-strap masks.

In some embodiments, the one or more connecting positions 124 for the upper-region strap and lower-region strap are elongated through-holes formed in the joining area between the PU and TPU layers. These through-holes are reinforced to prevent tearing and are located outside the flexible light panel 116, ensuring that light-emitting zones remain unobstructed during tightening.

In some embodiments, the dual-strap structure combined with the extended opening area 110 results in the mask body 100 that adheres uniformly to the user's skin, improves comfort, reduces light leakage near the eyes, and enhances the overall phototherapy effectiveness.

In some embodiments, the controller 102 is further provided with a global positioning system or GPS module capable of determining the user's real-time geographical location and time zone. The GPS module is electrically connected to the controller 102 within the controller 102 housing. The controller 102 periodically receives location coordinates, and based on the detected geographical position, the controller 102 automatically obtains the corresponding local time zone information.

In another embodiment, the controller 102 stores a therapy intensity database, and commands correlated optimal phototherapy by analysing parameters, such as wavelength range, light intensity, duty cycle, and duration, with environmental factors that vary from region to region. These factors include average sunlight exposure, ambient ultraviolet index, humidity levels, temperature range, and seasonal variations. By processing the GPS-derived location data, the controller 102 automatically selects a therapy profile suitable for the user's locale. For example, in regions with lower natural light exposure, the controller 102 may increase the LED brightness of the LED beads 118 or extend the duration of treatment. Conversely, in regions receiving intense sunlight, the controller 102 may reduce the therapy intensity to maintain safe and comfortable usage.

In another embodiment, the GPS module is also used to track time-zone changes when the user travels. Upon detecting a time-zone shift, the controller 102 automatically adjusts the therapy schedule to align with local circadian-rhythm requirements. The switch button 128 on the controller 102 is configured to activate an adaptive mode in which the microprocessor dynamically adjusts the light output of the flexible light panel 116 based on the GPS-derived environmental profile. This ensures consistent therapeutic effectiveness at any location without requiring user input.

In some embodiments, the GPS module is integrated with a low-power satellite synchronization circuit to minimize energy consumption. The controller 102 also includes a sleep mode in which GPS communication is periodically activated only when necessary, thereby prolonging battery life while maintaining accurate location-based therapy control. This GPS-enabled adaptive illumination capability delivers uniform intensity regardless of environmental or geographical variation.

In some embodiments, the controller 102 stores a therapy parameter database correlating geographic regions with recommended phototherapy settings. The database may include data fields associating latitude ranges with average ultraviolet index values, seasonal daylight duration, and typical ambient light exposure levels. When the GPS receiver determines the user's coordinates, the processor of the controller 102 queries the database to retrieve therapy parameters appropriate for the user's location. The controller 102 may automatically adjust the LED intensity of the LED beads 118, select appropriate wavelength combinations, and modify session duration based on the retrieved parameters. For example, users located at higher latitudes with reduced natural sunlight exposure may receive therapy sessions with increased LED intensity or extended duration, while users in equatorial regions with high ambient UV exposure may receive sessions with reduced intensity to avoid overstimulation. The controller 102 may also adjust therapy timing recommendations based on the local time zone to align treatment sessions with the user's circadian rhythm.

In an embodiment, the controller 102 incorporates an E-Ink or electrophoretic display, or digital ink display, mounted on the outer housing of the controller 102. The electrophoretic display is connected to the controller's 102 microprocessor and functions as a user interface for displaying operating status, therapy mode, battery percentage, and session timer information.

In another embodiment, the electrophoretic display operates based on electrophoretic particle migration and therefore consumes power only during screen refresh. During static display states, such as showing the therapy mode or time countdown, the display consumes essentially no electrical energy. This ultra-low power characteristic enables the controller 102 to achieve significantly prolonged battery life compared with controllers that employ conventional LCD or OLED screens.

In an embodiment, the electrophoretic display is configured to show several interface windows, including the current light-intensity level, selected wavelength program, local time, and real-time operating duration. The display also indicates the recommended therapy plan based on the GPS-adjusted settings. The use of an electrophoretic display also allows for high visibility under various lighting conditions, including bright indoor environments, making it easier for users to observe the mask body 100 status even during therapy sessions. The electrophoretic display is embedded flush with the controller 102 housing and protected by a transparent thermoplastic polyurethane or polycarbonate cover, preventing damage due to accidental impacts or daily handling. The controller 102, equipped with an electrophoretic interface, provides an energy-efficient, readable, and durable display solution that complements the flexible mask body 100 structure.

In an embodiment, a method of use for the phototherapy mask system is provided. The method enables a user to apply the phototherapy mask for facial treatment while achieving optimal facial conformity and uniform light delivery.

During use, the user threads a strap through the one or more connecting positions 124 disposed on both sides of each interior opening 112 along the width direction thereof. The strap is routed through the elongated through holes at the one or more connecting positions 124 on one side of the interior opening 112 and then through the corresponding one or more connecting positions 124 on the opposite side.

The user then tensions the strap by pulling the one or more connecting positions 124 on opposite sides of each interior opening 112 closer together along the width direction. This targeted tensioning draws the mask body 100 inward against the facial surface, substantially reducing bulging and minimizing the height differential between the mask body 100 and the user's skin. As the one or more connecting positions 124 are pulled together, the overlapping area of the flexible light panel 116 overlaps the clearance area 120, ensuring that no LED beads 118 are compressed or obstructed during the fit adjustment.

To initiate phototherapy, the user activates the controller 102 by pressing the switch button 128. Upon activation, the controller 102 supplies regulated electrical power and control signals through the connecting cable 126 to the flexible light panel 116 disposed within the mask body 100. The protective shell 130 at the junction between the connecting cable 126 and the mask body 100 maintains structural integrity during handling.

During the treatment session, the LED beads 118 on the flexible light panel 116 emit therapeutic light at specific wavelengths toward the user's facial skin. The translucent bottom film 114 allows the light to pass through to the skin, while the backlit top film 122 prevents light leakage from the rear side. The treatment continues for a predetermined duration and intensity as controlled by the controller 102.

In some embodiments, during use, the controller 102 equipped with a GPS receiver may automatically determine the user's location and adjust therapy parameters such as light intensity, therapy duration, or therapy timing based on the time zone or ambient sunlight conditions associated with the location. The user may view device status information, including therapy mode, intensity level, elapsed time, and battery status, on the electrophoretic display of the controller 102, which consumes power only when updating displayed content.

To end the treatment session, the user deactivates the system by pressing the switch button 128 on the controller 102. The controller 102 interrupts power delivery to the flexible light panel 116, thereby extinguishing the LED beads 118 and terminating the phototherapy cycle. The user then loosens the strap from the one or more connecting positions 124 and removes the mask body 100 from the face.

In some other embodiments, the present invention relates to a flexible phototherapy mask system configured to deliver controlled light-based therapy while conforming closely to the three-dimensional contours of a user’s face. The mask body incorporates a multilayer flexible structure in which a flexible light panel carrying a plurality of light-emitting elements is encapsulated between a translucent bottom film and a backlit top film. Strategically formed opening areas, interior openings, and reinforced connecting positions cooperate with adjustable straps to reduce bulging, improve facial conformity, and maintain uniform contact between the light-emitting surface and the skin. The structural arrangement of clearance areas and overlapping regions on the flexible light panel ensures that illumination elements are protected from mechanical stress during tightening, thereby enhancing durability and treatment reliability.

In some other embodiments, in use, the phototherapy mask is positioned over the user’s face such that the eye-region openings align with the user’s eyes and optional cutouts accommodate the nose and mouth. One or more straps are routed through the connecting positions disposed on opposite sides of the interior openings and are tightened to draw adjacent portions of the mask body inward along the width direction. This targeted tensioning allows the mask to closely follow facial contours across the forehead, eye region, cheeks, nose, and chin, minimizing air gaps and bulging effects commonly associated with conventional flexible masks. Upon activation of the controller, regulated electrical power is supplied to the flexible light panel, causing the light-emitting elements to emit therapeutic light toward the skin for a predetermined treatment duration and intensity.

In some other embodiments, the phototherapy mask system may be used for cosmetic, dermatological, and wellness-related facial treatments, including skin rejuvenation, complexion improvement, and targeted light therapy of specific facial regions. The flexible construction, dual-surface polymer films, and adaptive strap arrangement enable comfortable, prolonged wear while maintaining consistent optical output. The integration of a controller, optionally equipped with intelligent features such as location-based therapy adjustment and low-power display functionality, further enhances ease of use, treatment personalization, and operational efficiency.

The present invention finds industrial applicability in the cosmetics, dermatology, personal care, and wellness device industries. The phototherapy mask may be manufactured using scalable processes such as polymer film forming, flexible circuit assembly, ultrasonic welding, thermal welding, and electronic module integration, making it suitable for mass production. The device may be commercialized as a home-use facial phototherapy mask for personal skincare routines, a professional aesthetic treatment accessory for beauty practitioners, or a clinical dermatology device for therapeutic applications. The phototherapy mask system may be deployed in various facilities, including beauty centers, salons, spas, dermatology clinics, and medical facilities. The flexible architecture of the mask body, improved facial conformity through the strap-tensioned connecting positions, and robust encapsulation structure of the sealed multilayer light panel assembly provide significant advantages in user comfort, device durability, and therapeutic performance. The integration of intelligent features such as GPS-based adaptive therapy and low-power electrophoretic display technology further enhances the commercial viability and user experience of the phototherapy mask system.

Various modifications, adaptations, and variations of the embodiments described herein will be apparent to those skilled in the art in light of the foregoing disclosure. The principles of the present invention may be applied to other facial or wearable phototherapy devices without departing from the spirit and scope of the invention. Accordingly, the description is not intended to be limited to the specific embodiments disclosed but encompasses all alternatives, equivalents, and modifications falling within the scope of the appended claims.

1. A phototherapy mask, comprising:
a mask body being a flexible component, the mask body having two opening areas corresponding to eye regions of a user, each of the opening areas having an interior opening at one end away from the other opening area;
one or more connecting positions disposed on both sides of each interior opening along a width direction thereof, the connecting positions configured to receive a strap to pull the mask body closer together along the width direction of the interior opening when the strap is connected, thereby reducing bulging between the mask body and a facial surface of the user;
a flexible light panel disposed within the mask body, the flexible light panel comprising a substrate and a plurality of LED beads disposed on the substrate, wherein the substrate has a clearance area formed on one side of each interior opening along the width direction thereof and an overlapping area formed on an opposite side of each interior opening along the width direction, the overlapping area being configured to overlap the clearance area when two of the connecting positions are pulled together, the LED beads being located outside the clearance area; and
a controller electrically connected to the mask body, the controller configured to control the operation of the mask body.

2. The phototherapy mask of claim 1, wherein the mask body further comprises a translucent bottom film and a backlit top film, the translucent bottom film and the backlit top film being flexible layers, the translucent bottom film being located on a light-emitting side of the flexible light panel, and the backlit top film being located on a backlit side of the flexible light panel.

3. The phototherapy mask of claim 2, wherein areas of the translucent bottom film and the backlit top film are both larger than an area of the flexible light panel, and edges of the translucent bottom film and the backlit top film are connected to seal the flexible light panel, the connecting positions being disposed on an outside of the flexible light panel and located at a connection between the translucent bottom film and the backlit top film.

4. The phototherapy mask of claim 2, wherein the translucent bottom film is a thermoplastic polyurethane layer, and the backlit top film is a polyurethane layer.

5. The phototherapy mask of claim 4, wherein thickness of the translucent bottom film is 0.1mm to 0.9mm.

6. The phototherapy mask of claim 3, wherein the edges of the translucent bottom film and the backlit top film are connected by at least one of ultrasonic welding, thermal welding, or adhesive bonding.

7. The phototherapy mask of claim 1, wherein the one or more connecting positions are elongated through holes and the one or more connecting positions are located adjacently at two ends of the interior opening.

8. The phototherapy mask of claim 1, further comprising a connecting cable electrically connecting the controller and the mask body, and a protective shell disposed at a connection point between the connecting cable and the mask body, the protective shell configured to prevent the mask body from being torn under force.

9. The phototherapy mask of claim 1, wherein the mask body is provided with a mouth opening corresponding to the mouth of the user, and a nose opening corresponding to the nose of the user, the mask body having a nose phototherapy area disposed within the nose opening, a top of the nose phototherapy area being connected to the mask body.

10. A phototherapy mask system, comprising:
a mask body comprising a translucent bottom film, a flexible light panel, and a backlit top film, wherein the translucent bottom film and the backlit top film are flexible layers, the translucent bottom film is located on a light-emitting side of the flexible light panel, and the backlit top film is located on a backlit side of the flexible light panel;
wherein areas of the translucent bottom film and the backlit top film are larger than the area of the flexible light panel, and edges of the translucent bottom film and the backlit top film are connected to seal the flexible light panel;
wherein the mask body has two opening areas corresponding to eye regions of a user, each opening area having an interior opening, and connecting positions disposed on both sides of each interior opening along a width direction thereof, the connecting positions configured to receive a strap to pull the mask body closer together along the width direction of the interior opening when the strap is connected; and
a controller electrically connected to the mask body via a connecting cable, the controller comprising a housing, a control board, a battery, a switch button, and a GPS receiver, the GPS receiver configured to determine the location of the user and adjust therapy parameters based on the location.

11. The phototherapy mask system of claim 10, wherein the controller is configured to adjust at least one of light intensity, therapy duration, or therapy timing based on at least one of a time zone or ambient sunlight conditions associated with the location.

12. The phototherapy mask system of claim 10, wherein the flexible light panel comprises a substrate and a plurality of LED beads disposed on the substrate.

13. The phototherapy mask system of claim 12, wherein the substrate has a clearance area formed on one side of the interior opening along the width direction thereof, and the substrate has an overlapping area formed on another side of the interior opening along the width direction thereof, the overlapping area being configured to overlap the clearance area when two of the connecting positions overlap, the LED beads being located outside the clearance area.

14. The phototherapy mask system of claim 10, further comprising a protective shell disposed at a connection point between the connecting cable and the mask body, the protective shell configured to prevent the mask body from being torn under force.

15. The phototherapy mask system of claim 10, wherein the edges of the translucent bottom film and the backlit top film are connected by at least one of ultrasonic welding, thermal welding, or adhesive bonding.

16. A phototherapy mask, comprising:
a mask body being a flexible component having a translucent bottom film, a flexible light panel with a plurality of LED beads disposed on a substrate, and a backlit top film, wherein the translucent bottom film is a thermoplastic polyurethane layer, and the backlit top film is a polyurethane layer;
wherein the mask body has two opening areas corresponding to eye regions of a user, each opening area having an interior opening at one end away from the other opening area;
wherein the substrate has a clearance area formed on one side of each interior opening along a width direction thereof and an overlapping area formed on an opposite side of each interior opening along the width direction, the LED beads being located outside the clearance area, and the overlapping area being configured to overlap the clearance area when connecting positions on both sides of each interior opening are pulled together; and
a controller electrically connected to the mask body, the controller comprising a housing, a control board, a battery, a switch button, and an electrophoretic display configured to display device status information, the electrophoretic display consuming power only when updating displayed content.

17. The phototherapy mask of claim 16, wherein the device status information comprises at least one of a therapy mode, an intensity level, an elapsed time, or a battery status.

18. The phototherapy mask of claim 16, wherein the electrophoretic display maximizes battery life during treatment by not drawing power in a static display mode.

19. The phototherapy mask of claim 16, wherein areas of the translucent bottom film and the backlit top film are larger than the area of the flexible light panel, and edges of the translucent bottom film and the backlit top film are connected to seal the flexible light panel.

20. The phototherapy mask of claim 19, wherein the edges of the translucent bottom film and the backlit top film are connected by at least one of ultrasonic welding, thermal welding, or adhesive bonding.
